(19) United States
(12) Patent Application Publication
(10) Pub. No.: US 2022/0135909 A1
(43) Pub. Date: May 5, 2022

(54) STABLE ANHYDROUS DISINFECTANT CONCENTRATE FORMULATION AND METHOD OF MAKING THE SAME

(71) Applicant: One Home Brands, Inc., New York, NY (US)

(72) Inventors: Syed Humza NAQVI, Walnut Creek, CA (US); Patrick Shane Akeem HAREWOOD, West Haven, CT (US)

(73) Assignee: One Home Brands, Inc.

(21) Appl. No.: 17/506,476

(22) Filed: Oct. 20, 2021

Related U.S. Application Data
(60) Provisional application No. 63/106,998, filed on Oct. 29, 2020.

(51) Int. Cl. C11D 3/20; C11D 3/10; C11D 1/04; A61Q 17/00; A61K 8/34

(52) U.S. Cl. CPC C11D 3/2037; C11D 3/10; A61K 8/347; C11D 1/04; A61Q 17/005; C11D 3/2096

(57) ABSTRACT

The invention relates to stable, anhydrous concentrated disinfectant formulations.

### CROSS REFERENCE TO RELATED APPLICATIONS

This application claims priority to U.S. Provisional Application No. 63/106,998, filed Oct. 29, 2020, the entire contents of which are incorporated herein for reference.

### BACKGROUND

The majority of disinfectant products on the market are in liquid or gel forms and packaged in a plastic tube, bottle, spray bottle, or pump dispenser. The problem is the packaging. Single use plastic is everywhere and it is wreaking havoc on the environment. Only 9% of all plastic is actually recycled, and packaging generates the largest portion of municipal waste (˜30%). Packaged products are inefficient for businesses and the people who buy them.

Removing the water from disinfectant formulations removes the need for single use plastic packaging and the waste that comes with it, such as packaging waste, product waste, and the waste of resources used to ship water.

Thus, a need exists for new stable formulations of disinfectants that meet the needs of consumers, while also reducing the amount of waste generated in their production and shipping.

### SUMMARY OF THE INVENTION

The invention relates to stable anhydrous disinfectant cleanser concentrate formulations. The stable anhydrous disinfectant cleanser concentrate formulations may be in a solid form such as a tablet, granulars, powder, sachet, or polymer membrane (PVA, PVP, HPMC, etc) form. The solid stable anhydrous disinfectant cleanser concentrate formulations comprise a disinfectant, an acidic cleaner, a pH control agent which can be a basic cleaner, and an oily soil remover (a surfactant). The solid stable anhydrous disinfectant cleanser concentrate formulation may further comprise a binder. The solid stable anhydrous disinfectant cleanser concentrate formulation may further comprise at least one natural and/or synthetic fragrance. The solid stable anhydrous disinfectant cleanser concentrate formulation may further comprise a dye or coloring agent. The solid stable anhydrous disinfectant cleanser concentrate formulation may comprise other ingredients to aid the disinfectant process.

The solid stable anhydrous disinfectant cleanser concentrate formulation may be used as a disinfectant spray tablet or sachet, disinfectant multi-purpose spray cleaner tablet or sachet, disinfectant bathroom spray cleaner tablet or sachet, toilet bowl cleaner tablet sachet or tablet, spray or gel, disinfectant hand soap sachet or tablet, disinfectant wipes sachet or tablet, drain opener tablet or sachet, disinfectant laundry tablet or sachet. When diluted by the end user, the solid stable anhydrous disinfectant cleanser concentrate formulation may be used to clean hands, bathroom surfaces, kitchen surfaces, glass, multi-surface, and any other areas including but not limited to daily shower room, toilet bowl, floor, etc.

### DETAILED DESCRIPTION OF THE INVENTION

This disclosure relates to solid stable anhydrous disinfectant cleanser concentrate formulations. The inventors have discovered a solid disinfectant formulation that is both good for the environment and effective for cleaning purpose. The advantages of this solid disinfectant formulation over the traditional liquid disinfectant cleansers include chemical stability, reduced packaging due to lack of water in the formulation, reduced shipping weight due to lack of water in the formulation, and convenience for the consumer.

Specifically, the solid stable anhydrous disinfectant cleanser concentrate formulations of this disclosure may include a disinfectant, an acidic cleaner, a pH control agent (e.g., a basic cleaner), an oily soil remover (e.g., a surfactant), and an optional ingredient selected from a binder and a preservative. The formulation may optionally include one or more fragrances, dyes, or coloring agents.

As used in this specification, the singular forms “a,” “an,” and “the” include plural referents unless the context clearly dictates otherwise. Thus, for example, reference to a “preservative” includes a single kind of preservative or two or more different kinds of preservative.

“About” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. The term “about” when qualifying a value of a stated item, number, percentage, or term refers to a range of plus or minus ten percent of the value of the stated item, percentage, parameter, or term.

The term “oily soil remover” is used interchangeably with “surfactant.”

The term “anhydrous” as used herein refers to a stable, anhydrous disinfectant cleanser concentrate formulation comprising less than about 5%, 4%, 3%, 2% or 1% by weight of water based on the weight of the concentrate.

The term “substantially fatty acid-free” as used herein refers to a solid stable, anhydrous cleanser concentrate formulation comprising less than 5%, 4%, 3%, 2%, or 1% by weight of a fatty acid (or salt thereof) based on the weight of the concentrate, or comprising a fatty acid (or salt thereof) in an amount less than the amount used in a hand soap bar.

The term “substantially animal fat free” as used herein refers to a solid stable, anhydrous disinfectant cleanser concentrate formulation comprising less than 5%, 4%, 3%, 2%, or 1% by weight of an animal fat (such as tallow) (or salt thereof) based on the weight of the concentrate, or comprising or comprising a an animal fat (or salt thereof) in an amount less than the amount used in a hand soap bar.

Some of the ingredients may have multiple functions. However, when two or more ingredients defined based on their functions are included in a formulation disclosed herein, the ingredients differ from each other in terms of their chemical structure. For example citric acid can be a water softening agent and an acidic cleaner as well, but when both water softening agent and acidic cleaner are used in the description of the formulation, they intend to refer to different ingredients in terms of the chemical structure.

The term “comprising” includes the embodiments of “consisting of” or “consisting essentially of.”

The solid stable anhydrous disinfectant cleanser concentrate formulation includes a disinfectant to kill microorganisms, such as bacteria or viruses. Disinfectants known in the art to be compatible with solid compositions are contemplated by the present disclosure. Synthetic disinfectants may be used, however, in preferred embodiments the disinfectant is found in nature. In preferred embodiments the disinfectant is extracted from a plant or plant product. The disinfectant may by thymol. Thymol is a component of thyme oil and is similar to the plant extract. Thyme oil is a minimal risk pesticide and has low toxicity. Thyme oil may be incorporated as part of an essential oil or fragrance. Thymol works as a disinfectant by inducing instability in a microorganism's membrane, causing cell content leakage and death of the microorganism. Thymol crystals can also be synthetically made.

The amount of thymol in the solid stable anhydrous disinfectant cleanser concentrate formulation may range from about 1% to about 40% by weight. The amount of disinfectant per formulation may be about 1%, about 5%, about 10%, about 15%, about 20%, about 25%, about 30%, about 40%, about 5% to about 40%, about 10% to about 35%, about 10% to about 30%, about 15% to about 25%, about 20% to about 25% by weight of the formulation.

After the solid stable anhydrous disinfectant cleanser concentrate formulation is diluted in water, the concentration of thymol will be reduced to a lower amount, such as about 0.05% to about 0.30% by weight. For example, if the thymol concentration of 5% by weight in a 8.0 gram solid stable anhydrous disinfectant cleanser concentrate tablet (about 0.40 grams of thymol) is diluted in 24 ounces of water (which is about 710 grams of water), the thymol concentration is reduced to about 0.056% by weight. As another example, if the thymol concentration of 22.5% by weight in a 8.0 gram solid stable anhydrous disinfectant cleanser concentrate formulation (about 1.64 grams of thymol) is diluted in 24 ounces of water (which is about 710 grams of water), the thymol concentration is reduced to about 0.23% by weight.

In some examples, the solid stable anhydrous disinfectant cleanser concentrate formulation may also include copper sulfate (bluestone). The copper sulfate may help the thymol work properly as a disinfectant. Copper sulfate has properties as disinfectant. The amount of copper sulfate in the solid stable anhydrous disinfectant cleanser concentrate formulation may range from about 0% to about 20% by weight, such as about 0%, about 5%, about 10%, by weight of the formulation. When included, the copper sulfate may be in a ratio between 10:1 to 1:10 with thymol, e.g., between 7:1 and 1:7, between 5:1 and 1:5, between 3:1 and 1:3, between 7:1 and 1:1, between 5:1 and 1:1, between 3:1 and 1:1, between 1:1 and 1:3, between 1:1 and 1:5, between 1:1 and 1:7, between 1:1 and 1:10, between 10:1 and 9:1, between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 1:1, between 1:1 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, between 1:9 and 1:10, between 10:1 and 5:1, between 5:1 and 1:1, or between 1:5 and 1:10. In a preferred embodiment, the copper sulfate is in a ratio of about 2:1 with the thymol (copper sulfate:thymol). After the solid stable anhydrous disinfectant cleanser concentrate formulation is diluted in water, the concentration of copper sulfate will be reduced to a lower amount, such as reduced to 0.10%.

In some examples, the solid stable anhydrous disinfectant cleanser concentrate formulation may include sorbitan caprylate. The sorbitan caprylate may help the thymol work properly as a disinfectant. The sorbitan caprylate may help disturb the microorganism cell walls, which allows other ingredients to enter the microorganism and kill it. The sorbitan caprylate may also act as a preservative booster by helping to kill microorganisms that would harm the formulation. The amount of sorbitan caprylate in the solid stable anhydrous disinfectant cleanser concentrate formulation may range from about 1% to about 5% by weight, such as about 0.1%, about 1%, about 0.5%, by weight of the formulation.

The amount of acidic cleaner in the solid stable anhydrous disinfectant cleanser concentrate formulation may range from about 1% to about 50% by weight, based on the weight of the formulation. The amount of acidic cleaner per tablet may be about 1%, about 5%, about 10%, about 15%, about 20%, about 25%, about 30%, about 40%, about 50%, about 5% to about 50%, about 10% to about 45%, about 15% to about 40%, about 20% to about 35%, about 30% to about 35%, by weight of the formulation. The acidic cleaner may be those known in the art to be compatible with solid cleaning formulations. Though the acidic cleaner may be synthetic, it is preferred that the acidic cleaner be found in nature (e.g., extractable from a plant). Preferably the acidic cleaner is readily biodegradable. Exemplary biodegradable acids include organic carboxylic acids, e.g., aromatic and heteroaromatic carboxylic acids, alkyl carboxylic acids (e.g., C₃-C₈ carboxlic acids), heterocyclic carboxylic acids, including molecules with one, two, or three carboxylic acid moieties). Exemplary carboxylic acids include, but are not limited to, citric acid, malic acid, fumaric acid, etc. The acidic cleaner may be citric acid. The citric acid may be a particular type of citric acid that is coated. The coated citric acid may aid in stability of the formulation. The coated citric acid may be coated so it will not react to base of the formulation, which may make it more stable tablet formula in humid environments. For example, the coated citric acid may be Jungbunzlauer Citrocoat N10, which is 90% citric acid and 10% sodium citrate. Other possible coated citric acids may include Citrocoat N, which is 97.5% citric acid and 2.5% sodium citrate or Citrocoat DC, which is citric acid coated with maltodextrin. The acidic cleaner may also be malic acid, fumaric acid, acetic acid, sodium acetate (salt), and/or glycolic acid.

The solid stable anhydrous disinfectant cleanser concentrate formulation may contain a pH control agent in an amount sufficient to adjust the pH when dissolved in water from about 2.0 to about 12.5, from about 5.0 to about 6.0, from about 3.5 to about 4.5, from about 4.0 to about 5.0, from about 4.5 to about 5.5, or from about 9.5 to about 10.5. The pH of the dissolved tablet in water may be about 2.0, about 2.5, about 3.0, about 3.5, about 4.0, about 4.5, about 5.0, about 5.5, about 2.0 to about 5.5, about 7.5, about 8.0, about 8.5, about 9.0, about 10.0, about 10.5, about 11.0, about 11.5, about 12.0, about 12.5, or about 7.5 to about 12.5. The pH control agent may be any agent sufficient to raise or lower the pH of the tablet when dissolved in water, which includes a basic cleaner and an acidic cleaner, such as sodium carbonate, sodium bicarbonate, citric acid, or malic acid. When both an acidic cleaner and a pH control agent are contained in the solid formulation, the acidic cleaner differs from the pH control agent. An acidic pH of the solid stable anhydrous disinfectant cleanser concentrate formulation may also help act as a disinfectant by helping to kill microorganisms.

When the pH control agent is a basic cleaner, the amount of the basic cleaner may range from about 5% to about 60%, from about 5% to about 30%, from about 10% to about 30%, from about 10% to about 25%, from about 5% to about 10%, from about 40% to about 60%, or from about 35% to about 45%, by weight, based on the weight of the formulation. The basic cleaner may be sodium carbonate, sodium bicarbonate and/or any other alkali carbonates.

The acidic cleaner and basic cleaner together are referred as effervescent ingredients. In one aspect, the solid stable anhydrous disinfectant cleanser concentrate formulation can comprise the effervescent ingredients in an amount ranging from about 30% to about 80 or from about 50% to about 55% by weight. The effervescent ingredients (e.g, the acidic and basic cleaner) can facilitate homogeneous distribution and dissolution of the surfactant into water before use.

The amount of a binder in the solid stable, anhydrous disinfectant cleanser concentrate formulation may range from about 0 to about 50% such as about 1% to about 20%, less than about 5%, from about 0% to about 5%, from about 3% to about 7%, from about 4% to about 8%, by weight, based on the weight of the formulation. The amount of binder may be about 1%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 2% to about 10.0%, about 3% to about 7%, about 1% to about 10%, or about 4% to about 8% by weight. The amount of binding agent in the solid stable, anhydrous cleanser concentrate formulation may range from about 1% to about 20% by weight, based on the weight of the formulation. The amount of binding agent is suitable to form a tablet and may be about 1%, about 2%, about 3%, about 4%, about 5%, about 6%, about 7%, about 8%, about 9%, about 10%, about 12%, about 15%, about 18%, about 20%, about 3% to about 7%, about 4% to about 8%, about 5% to about 10%, or about 10% to about 20%. The solid stable, anhydrous cleanser concentrate formulation may contain one or more binders selected from the group consisting of hydrated dextrate (e.g., EMDEX), polyethylene glycol (e.g., polyethylene glycol 8000), dextrose, microcrystalline cellulose, and maltodextrin, pullulan, starch, polyvinylpyrrolidone.

The amount of oily soil remover (a surfactant) in the solid stable, anhydrous cleanser concentrate formulation may range from 0.01% to about 40% such as from about 1% to about 20%, from about 2% to about 15%, from about 8% to about 12%, from about 1% to about 15%, from about 3% to about 7%, from about 6% to about 20%, from about 16% to about 20%, or from about 10% to 14% by weight, based on the weight of the formulation. The amount of oily soil remover may be about 1%, about 2%, about 3%, about 4%, about 5%, about 6%, about 7%, about 8%, about 9%, about 10%, about 12%, about 15%, about 18%, about 20%, about 16% to about 20%, about 3% to about 7%, about 4% to about 8%, about 8% to about 12%, about 5% to about 10%, about 10% to about 15%, or about 10% to about 20%. The oily soil remover (i.e. surfactant) can be natural or synthetic surfactants, such an anionic, nonionic, amphoteric, zwitterioinic, or cationic surfactants, such as anionic and non-ionic surfactants, further such as a surfactant selected from sodium coco sulfate, ethoxylated alcohols (such as ethoxylated alcohol C(10-12)-C(14-16) with 4-8 moles ethoxylation, for example Clariant Genapol LA 060 (ethoxylated alcohol C12-C16) w/6 moles ethoxylation, ethoxylated alcohols C8-C10 6-8 moles of EO, etc.), sodium lauryl sulfate, sorbitan caprylate, cationic quaternary ammonium, and alkyl polyglucosides (such as lauryl glucoside, caprylyl/myristyl glucoside, caprylyl/decyl Glucoside).

The solid stable anhydrous disinfectant cleanser concentrate formulation may include a preservative. The amount of the preservative in the solid stable, anhydrous cleanser concentrate formulation may range from about 5% to about 40%, from 5% to about 30%, from about 10% to about 30%, from about 10% to about 25%, or from about 10% to about 20%, by weight, based on the weight of the formulation. The amount of the preservative may be about 5%, about 7%, about 9%, about 11%, about 13%, about 15%, about 17%, about 19%, about 21%, about 23%, about 25%, about 27%, about 29%, about 31%, about 33%, about 35%, about 37%, about 39%, or about 40%. The preservative may be sodium benzoate, potassium sorbate, gluconolactone, and/or biocideal preservatives. In some examples, sodium benzoate may also work as process aid for processing the powder to make the formulation into tablets.

The amount of the preservative booster in the solid stable anhydrous disinfectant cleanser concentrate formulation may range from about 0.1% to about 15%, from about 0.5% to about 10%, from about 1% to about 10%, or from about 1% to about 5%, by weight, based on the weight of the formulation. The amount of the preservative booster may be about 0.1%, about 0.2%, about 0.3%, about 0.4%, about 0.5%, about 1%, about 2%, about 3%, about 4%, about 5%, about 6%, about 7%, about 8%, about 9%, or about 10%. Preservative boosters compatible with the present disclosure include those known in the art to aid in the suppression of bacterial and/or fungal growth by preservatives. Preservative boosters include, but are not limited to, sodium benzoate, potassium sorbate, sorbitan caprylate, and gluconolactone. In a preferred embodiments, the preservative booster is a sorbate such as potassium sorbate.

The solid stable anhydrous disinfectant cleanser concentrate formulation may contain one or more fragrances, such as natural fragrances (e.g., essential oils) and/or synthetic fragrances and perfumes in the form of oils, crystals, powders, granules, and encapsulations. The thymol in the formulation may partially come from thyme oil added as a fragrance.

The solid stable anhydrous disinfectant cleanser concentrate formulation may contain one or more dyes or coloring agents, such as Food, Drug and Cosmetic (FD&C) approved dyes and colorants.

In some embodiments the solid stable anhydrous disinfectant cleanser concentrate formulation may include silica as a flow aid for the formation of free-flowing powder. Free-flowing powder is subsequently converted into tablets.

In some embodiments, the solid stable anhydrous disinfectant cleanser concentrate formulation does not include silica. The precursor such as free-flowing powder can be formed by using solids, such as sodium carbonate and sodium benzoate, as the initial ingredients to absorb any potential liquids that can be used in the cleaner concentrate formulation.

In some embodiments, the solid stable anhydrous disinfectant cleanser concentrate formulation may be formed into a tablet that is dilutable in water. In other embodiments, the solid stable anhydrous disinfectant cleanser concentrate formulation may be other forms, such as a sachet with powder or crystals, powder with membrane (PVA, HPMC, PVP, pullulan, etc), wet granulation, spray dried powder in sachet or tablet, liquid concentrate to dilute, pod type with powder or liquid concentrate, or gel concentrate.

When the solid stable anhydrous disinfectant cleanser concentrate formulation is in the form of a tablet. The tablet can be in any size. For example, it may weigh from about 2.0 grams to about 18 grams, such as from about 4.5 grams to about 5.5 grams, such as from about 6.5 grams to about 7.1 grams, from about 6.0 grams and about 7.5 grams, from about 7.9 grams to about 8.5 grams, about 2.5 grams, about 3 grams, about 4 grams, about 5 grams, about 6 grams, about 7 grams, about 8 grams or more preferably about 9 grams. In a preferred embodiment, the tablets are round, however other geometric shapes are contemplated.

The tablet can be dissolved into appropriate amount of water before use. For example, the ratio of tablet to water can range from 5-12 grams of tablets in 24-34 ounces of water, or more preferably about 8.0 grams of tablets to 24 ounces of water, 9.33 grams of tablets to 28 ounces of water, 10.66 grams of tablets to 32 ounces of water, or 11.33 grams of tablets to 34 ounces of water.

In each of the preceding embodiments, the solid stable anhydrous disinfectant cleanser concentrate formulation can be substantially fatty acid free and/or substantially animal fat free.

The solid stable anhydrous disinfectant cleanser concentrate formulations may be a disinfectant spray tablet or sachet, disinfectant multi-purpose spray cleaner tablet or sachet, disinfectant bathroom spray cleaner tablet or sachet, toilet bowl cleaner tablet sachet or tablet, spray or gel, disinfectant hand soap sachet or tablet, disinfectant wipes sachet or tablet, drain opener tablet or sachet, disinfectant laundry tablet or sachet.

The solid stable anhydrous disinfectant cleanser concentrate formulations may be diluted by the end user at their home in order to create the final diluted formulation. The final diluted formulation may be used to clean hands, bathroom, glass, multi-surface, and any other areas including but not limited to daily shower room, toilet bowl, floor, etc.

### Methods for Preparing Stable Anhydrous Disinfectant Cleanser Tablets

The solid stable anhydrous disinfectant cleanser tablets can be prepared using any suitable method. Stable anhydrous disinfectant cleanser tablet can be prepared using direct compression or wet granulation process. The term direct compression (or direct compaction) is used to define the process by which tablets are compressed directly from powdered substance and suitable excipients into a firm compact without employing the process of granulation. Powder is blended homogeneously using a blender (Ribbon Blender, V-blender, paddle blender, drum mixing). The powder blender is then charged into the hopper of tablet press (such as a Stokes DS-3). Desire weight, compression ton, and hardness of tablet are set as the tablets get compressed and come out of the tablet press.

To avoid effervescence from happening during storage, the disinfectant cleanser formulation can be pressed to achieve a sufficient hardness and/or the formulation contains a desiccating agent such as hydrated silica or any other agent known to absorb moisture. In preferred embodiments, the thymol is added to the formulation as a solid (e.g., crystalline) rather than as thyme oil.

### Format

The stable anhydrous disinfectant cleanser concentrate formulations in powder form can also be diluted in water in a powder to water ratio of greater than or equal to 1:1 (w/w) to form a paste before shipment. A method of using the paste comprises placing the paste on surface to be cleaned either directly or through a rag or sponge, optionally leaving overnight to soak, and rinsing the surface water.

The solid stable anhydrous disinfectant cleanser concentrate formulations described herein may be designed to be rinsed off, wiped, off, or left off for maximum cleaning efficiency.

### Methods of Using Stable Anhydrous Disinfectant Cleanser Tablets

In one aspect, disclosed is a method of using any of the tablets described herein including the steps of (1) filling a spray bottle or vessel with volume of 16-34 ounces with water, (2) adding the disinfectant cleaning tablet to the water-filled spray bottle, and (3) dissolving the tablet in water by no stirring or shaking required. In some embodiments, one or more disinfectant cleaning tablets may be added to the water-filled spray bottle. For example, two disinfectant cleaning tablets may be added to the spray bottle simultaneously or in a row before ultimately using the liquid solution for disinfectant cleaning purpose.

In some embodiments, when employed as described above, the cleanser tablet has a dissolution time of less than less than 25 minutes, e.g., less than 20 minutes, less than 15 minutes, less than 10 minutes, less than 8 minutes, less than 6 minutes, or less than 4 minutes.

Each individual disinfectant cleanser tablet, when exposed to water, will dissolve into a liquid cleansing solution. Upon experiencing dissolution of the disinfectant cleanser tablet, the user may proceed with disinfectant cleaning or washing as usual. Individual tablets may be packaged together in suitable bulk quantities.

The disinfectant cleanser tablets may be stored in any suitable container, such as but not limited to paper, foil, plastic, glass, aluminum, ceramic, acrylic, or copolyester (Eastman Tritan) containers. The container may contain a desiccant. The container may be re-usable and refilled with new tablets as needed.

### EXEMPLIFICATION

All the disclosed aspects of the stable anhydrous disinfectant cleanser concentrate formulations may be used with or applied to any of the examples listed.

### Example 1

TABLE 1

| Ingredient | Weight (%) | Weight (g) |
|---|---|---|
| Sodium Carbonate (Base) | 18.00 | 1.44 |
| Fragrance | 1.50 | 0.12 |
| Sorbitan Caprylate (Nonionic Surfactant) | 0.50 | 0.04 |
| Sodium Benzoate (Preservative) | 14.00 | 1.12 |
| Potassium Sorbate (Preservative) | 4.00 | 0.32 |
| Hydrated Dextrate (Binder) | 5.00 | 0.40 |
| Sodium Lauryl Sulfate (Anionic Surfactant) | 6.00 | 0.48 |
| Thymol Crystals = >0.36 gram/725 g | 5.00 | 0.40 |
| (Active Ingredient) | | |
| Copper Sulfate (bluestone) = 2X Thymol | 10.00 | 0.80 |
| Citric Acid (Citrocoat N10) (Acid) | 36.00 | 2.88 |
| Total | 100.00 | 8.00 |

Example 1 may be formed into a solid tablet weighing 8.0 grams. Example 1 may have a pH of about 3.0. Due to the relatively low pH, Example 1 may preferably be used as a bathroom cleaner and/or a tub and tile cleaner. Example 1 may have 0.05% thymol in the final diluted liquid disinfectant cleansing solution when diluted with about 24 ounces of water. Example 1 may include copper sulfate to aid the thymol working as a disinfectant.

Additional exemplary cleaner formulations with relatively low pH that may be used as a bathroom cleaner and/or a tub and tile cleaner are shown in Table 2.

TABLE 2

| | Low pH | | Low pH | | Low pH | |
|---|---|---|---|---|---|---|
| | Formulation A | | Formulation B | | Formulation C | |
| | Weight | Weight | Weight | Weight | Weight | Weight |
| Ingredient | (%) | (g) | (%) | (g) | (%) | (g) |
| Sodium Carbonate | 20.00 | 1.60 | 18.50 | 1.48 | 18.00 | 1.44 |
| (Base) | | | | | | |
| Fragrance | 2.00 | 0.16 | 2.00 | 0.16 | 2.00 | 0.16 |
| Sorbitan Capylate | 0.50 | 0.04 | 0.50 | 0.04 | 0.50 | 0.04 |
| (Nonionic Surfactant- | | | | | | |
| Fragrance emulsifier) | | | | | | |
| Sipernat 50 | 1.00 | 0.08 | 1.00 | 0.08 | 1.00 | 0.08 |
| (Silica-drying agent) | | | | | | |
| Sodium Gluconate | 23.50 | 1.88 | 17.55 | 1.40 | 13.50 | 1.08 |
| (chelting agent/ | | | | | | |
| q.s agent/Filler) | | | | | | |
| Hydrated Dextrate | 5.00 | 0.40 | 5.00 | 0.40 | 5.00 | 0.40 |
| (Binder) | | | | | | |
| Sodium Lauryl Sulfate | 5.00 | 0.40 | 5.00 | 0.40 | 5.00 | 0.40 |
| (Anionic Surfactant- | | | | | | |
| dirt, grime, and grease remover) | | | | | | |
| Sodium Coco Sulfate-granulated, | 4.00 | 0.32 | 4.00 | 0.32 | 4.00 | 0.32 |
| (Anionic Surfactant- | | | | | | |
| dirt, grime, and grease remover) | | | | | | |
| Thymol Crystals | 5.00 | 0.40 | 5.00 | 0.40 | 5.00 | 0.40 |
| (Disinfectant Active agent) | | | | | | |
| Copper Sulfate | 10.00 | 0.80 | 10.00 | 0.80 | 10.00 | 0.80 |
| (helps disinfect) | | | | | | |
| Coated Citric Acid | 24.00 | 1.92 | 31.45 | 2.52 | 36.00 | 2.88 |
| (Citrocoat N10) | | | | | | |
| Total | 100.00 | 8.00 | 100.00 | 8.00 | 100.00 | 8.00 |
| Tablet Size | | 8.00 | | 8.00 | | 8.00 |
| pH | 4.3-4.6 | | 3.9-4.2 | | 3.0-3.3 | |

### Example 2

TABLE 3

| Ingredient | Weight (%) | Weight (g) |
|---|---|---|
| Sodium Carbonate (Base) | 23.00 | 1.84 |
| Fragrance | 1.50 | 0.12 |
| Sorbitan Caprylate (Nonionic Surfactant) | 0.50 | 0.04 |
| Sodium Benzoate (Preservative) | 14.00 | 1.12 |
| Potassium Sorbate (Preservative) | 4.00 | 0.32 |
| Hydrated Dextrate (Binder) | 5.00 | 0.40 |
| Sodium Lauryl Sulfate (Anionic Surfactant) | 6.00 | 0.48 |
| Thymol Crystals = >0.36 gram/725 g | 5.00 | 0.40 |
| (Active Ingredient) | | |
| Copper Sulfate (bluestone) = 2X Thymol | 10.00 | 0.80 |
| Citric Acid (Citrocoat N10) (Acid) | 31.00 | 2.48 |
| Total | 100.00 | 8.00 |

Example 2 may be formed into a solid tablet weighing 8.0 grams. Example 2 may have a pH of about 5-6. Due to the pH, Example 2 may be used as a cleaner for multiple surfaces. Example 2 may have 0.05% thymol in the final diluted liquid disinfectant cleansing solution when diluted with about 24 ounces of water. Example 2 may include copper sulfate to aid the thymol working as a disinfectant.

Additional exemplary cleaner formulations with moderate pH that may be used for multiple surfaces are shown in Table 4.

TABLE 4

| | Moderate pH | | Moderate pH | | Moderate pH | |
|---|---|---|---|---|---|---|
| | Formulation A | | Formulation B | | Formulation C | |
| | Weight | Weight | Weight | Weight | Weight | Weight |
| Ingredient | (%) | (g) | (%) | (g) | (%) | (g) |
| Sodium Carbonate | 35.00 | 2.80 | 30.00 | 2.40 | 25.00 | 2.00 |
| (Base) | | | | | | |
| Fragrance | 2.00 | 0.16 | 2.00 | 0.16 | 2.00 | 0.16 |
| Sorbitan Capylate | 0.50 | 0.04 | 0.50 | 0.04 | 0.50 | 0.04 |
| (Nonionic Surfactant- | | | | | | |
| Fragrance emulsifier) | | | | | | |
| Sipernat 50 | 1.00 | 0.08 | 1.00 | 0.08 | 1.00 | 0.08 |
| (Silica-drying agent) | | | | | | |
| Sodium Gluconate | 7.50 | 0.60 | 12.50 | 1.00 | 17.50 | 1.40 |
| (chelting agent/ | | | | | | |
| q.s agent/Filler) | | | | | | |
| Hydrated Dextrate | 5.00 | 0.40 | 5.00 | 0.40 | 5.00 | 0.40 |
| (Binder) | | | | | | |
| Sodium Lauryl Sulfate | 5.00 | 0.40 | 5.00 | 0.40 | 5.00 | 0.40 |
| (Anionic Surfactant- | | | | | | |
| dirt, grime, and grease remover) | | | | | | |
| Sodium Coco Sulfate-granulated, | 4.00 | 0.32 | 4.00 | 0.32 | 4.00 | 0.32 |
| (Anionic Surfactant- | | | | | | |
| dirt, grime, and grease remover) | | | | | | |
| Thymol Crystals | 5.00 | 0.40 | 5.00 | 0.40 | 5.00 | 0.40 |
| (Disinfectant Active agent) | | | | | | |
| Copper Sulfate | 10.00 | 0.80 | 10.00 | 0.80 | 10.00 | 0.80 |
| (helps disinfect) | | | | | | |
| Coated Citric Acid | 25.00 | 2.00 | 25.00 | 2.00 | 25.00 | 2.00 |
| (Citrocoat N10) | | | | | | |
| Total | 100.00 | 8.00 | 100.00 | 8.00 | 100.00 | 8.00 |
| Tablet Size | | 8.00 | | 8.00 | | 8.00 |
| pH | 5.8-6.0 | | 5.3-5.5 | | 4.9-5.1 | |

### Example 3

TABLE 5

| Ingredient | Weight (%) | Weight (g) |
|---|---|---|
| Sodium Carbonate (Dense) | 18.50 | 1.48 |
| Fragrance | 1.50 | 0.12 |
| Sorbitan Caprylate (Nonionic Surfactant) | 0.50 | 0.04 |
| Sodium Benzoate (Preservative) | 10.55 | 0.84 |
| Potassium Sorbate (Preservative) | 4.00 | 0.32 |
| Hydrated Dextrate (Binder) | 5.00 | 0.40 |
| Sodium Lauryl Sulfate (Anionic Surfactant) | 6.00 | 0.48 |
| Thymol Crystals = 1.80 g/725 g of | 22.50 | 1.80 |
| water > 0.23% | | |
| Active Thymol | | |
| Citric Acid | 31.45 | 2.52 |
| Total | 100.00 | 8.00 |

Example 3 may be formed into a solid tablet weighing 8.0 grams. Example 3 may include a higher concentration of thymol than Examples 1 or 2. Example 3 may have about 0.23% thymol in the final diluted liquid disinfectant cleansing solution when diluted with about 725 grams of water. Example 3 may not include copper sulfate due to the relatively high concentration of thymol in Example 3. Instructions for dilution of an Example 3 tablet may state to use 24 ounces of water, which is about 710 grams, but if the end user dilutes an Example 3 tablet in more than 24 ounces of water, such as 725 grams of water, an Example 3 tablet will include enough thymol for the final diluted liquid disinfectant cleansing solution to be an effective disinfectant.

An additional exemplary cleaner formulation with a relatively higher concentration of thymol is shown in Table 6.

TABLE 6

| | High Thymol | |
|---|---|---|
| | Formulation A | |
| | Weight | Weight |
| Ingredient | (%) | (g) |
| Sodium Carbonate (Base) | 18.50 | 1.48 |
| Fragrance | 2.00 | 0.16 |
| Sorbitan Capylate (Nonionic Surfactant- | 0.50 | 0.04 |
| Fragrance emulsifier) | | |
| Sipernat 50 (Silica-drying agent) | 1.00 | 0.08 |
| Sodium Gluconate (chelting agent/q.s agent/Filler) | 11.13 | 0.89 |
| Hydrated Dextrate (Binder) | 5.00 | 0.40 |
| Sodium Lauryl Sulfate (Anionic Surfactant- | 6.00 | 0.48 |
| dirt, grime, and grease remover) | | |
| Sodium Coco Sulfate-granulated, | 4.00 | 0.32 |
| (Anionic Surfactant- | | |
| dirt, grime, and grease remover) | | |
| Thymol Crystals (Disinfectant Active agent) | 20.42 | 1.63 |
| Copper Sulfate (helps disinfect) | 0.00 | 0.00 |
| Coated Citric Acid (Citrocoat N10) | 31.45 | 2.52 |
| Total | 100.00 | 8.00 |
| Tablet Size | | 8.00 |
| pH | 4.4-4.6 | |

Notes:
1.633 g Thymol/710 g (24 oz) of water = 0.23% Active

The disclosed aspects of the stable anhydrous disinfectant cleanser concentrate formulations may also be used in disinfectant formulations that are not designed to be cleansers or that do not function as cleansers. The stable anhydrous disinfectant concentrate formulations may include relatively low amounts of surfactants and may have a safe pH range to allow use of the disinfectant as a spray disinfectant on any surface.

Exemplary stable anhydrous disinfectant concentrate formulations are shown in Table 7.

TABLE 7

| | Disinfectant | | Disinfectant | | Disinfectant | |
|---|---|---|---|---|---|---|
| | Formulation A | | Formulation B | | Formulation C | |
| | Weight | Weight | Weight | Weight | Weight | Weight |
| Ingredient | (%) | (g) | (%) | (g) | (%) | (g) |
| Sodium Carbonate | 35.00 | 2.80 | 30.00 | 2.40 | 25.00 | 2.00 |
| (Base) | | | | | | |
| Fragrance | 2.00 | 0.16 | 2.00 | 0.16 | 2.00 | 0.16 |
| Sorbitan Capylate | 0.50 | 0.04 | 0.50 | 0.04 | 0.50 | 0.04 |
| (Nonionic Surfactant- | | | | | | |
| Fragrance emulsifier) | | | | | | |
| Sipernat 50 | 1.00 | 0.08 | 1.00 | 0.08 | 1.00 | 0.08 |
| (Silica-drying agent) | | | | | | |
| Sodium Gluconate | 13.50 | 1.08 | 18.50 | 1.48 | 23.50 | 1.88 |
| (chelting agent/ | | | | | | |
| q.s agent/Filler) | | | | | | |
| Hydrated Dextrate | 5.00 | 0.40 | 5.00 | 0.40 | 5.00 | 0.40 |
| (Binder) | | | | | | |
| Sodium Lauryl Sulfate | 3.00 | 0.24 | 3.00 | 0.24 | 3.00 | 0.24 |
| (Anionic Surfactant- | | | | | | |
| dirt, grime, and grease remover) | | | | | | |
| Sodium Coco Sulfate-granulated, | 0.00 | 0.00 | 0.00 | 0.00 | 0.00 | 0.00 |
| (Anionic Surfactant- | | | | | | |
| dirt, grime, and grease remover) | | | | | | |
| Thymol Crystals | 5.00 | 0.40 | 5.00 | 0.40 | 5.00 | 0.40 |
| (Disinfectant Active agent) | | | | | | |
| Copper Sulfate | 10.00 | 0.80 | 10.00 | 0.80 | 10.00 | 0.80 |
| (helps disinfect) | | | | | | |
| Coated Citric Acid | 25.00 | 2.00 | 25.00 | 2.00 | 25.00 | 2.00 |
| (Citrocoat N10) | | | | | | |
| Total | 100.00 | 8.00 | 100.00 | 8.00 | 100.00 | 8.00 |
| Tablet Size | | 8.00 | | 8.00 | | 8.00 |
| pH | 5.8-6.0 | | 5.3-5.5 | | 4.9-5.1 | |

### Example 4

TABLE 8

| | Disinfectant Formulation | |
|---|---|---|
| | | Weight |
| Ingredient | | (%) |
| Binder (Dextrate) | EMDEX | 10.00 |
| Surfactant | Texapon K12 P or | 6.00 |
| (Coco sulfate powder) | Stepanol ME DRY | |
| Surfactant | Sulfopon 1216 G | 3.00 |
| (Coco sulfate granular) | | |
| Disinfectant Active | Thymol Crystals | 4.50 |
| Disinfectant Active | Cupric Sulfate | 9.00 |
| | Pentahydrate Technical | |
| Effervescent Acid (Citric Acid) | CITROCOAT ® N10 | 42.00 |
| Surfactant (Sorbital derived) | Sorbitan Caprylate | 1.55 |
| Fragrance | Fragrance Fresh Scent | 1.00 |
| Effervescent Base | Soda Ash | 22.95 |
| (Sodium Carbonate) | | |
| | Total | 100.00 |

Example 4 demonstrates one functional example of a stable anhydrous disinfectant cleanser formulation. In this example, the formulation is prepared with thymol crystals rather than a liquid composition. Additionally, the ratio of cupric sulfate pentahydrate to thymol crystals is 2:1 in this example.

### Example 5

TABLE 9

| | Disinfectant Formulation | |
|---|---|---|
| Ingredient type | | % |
| Binder (Dextrate) | EMDEX | 17.50 |
| Surfactant | Texapon K12 P or | 6.00 |
| (Coco sulfate powder) | Stepanol ME DRY | |
| Surfactant (Coco sulfate granular) | BASF Sulfopon 1216 G | 3.00 |
| Disinfectant Active | Thymol Crystals | 4.50 |
| Disinfectant Active | Cupric Sulfate | 0.74 |
| | Pentahydrate Technical | |
| Effervescent Acid (Citric Acid) | CITROCOAT ® N10 | 38.75 |
| Surfactant (Sorbital derived) | Sorbitan Caprylate | 1.51 |
| Fragrance | Fragrance - Pink Grapefruit | 1.00 |
| Effervescent Base | Soda Ash | 27.00 |
| (Sodium Carbonate) | | |
| | Total | 100.00 |

Example 5 discloses a stable anhydrous disinfectant cleanser concentrate formulation. In this example, the formulation was prepared with solid thymol crystals. In this example, the ratio of cupric sulfate pentahydrate to thymol is approximately 1:6.

### Example 6

Cleanser Embodiment-Multi-Surface Disinfectant

| | | Number Confirmed | | | | |
|---|---|---|---|---|---|---|
| Organic | | Positive Carriers/ | | | | |
| Soil | | Total Number Tested | | | | |
| Load | Microbe(s) | Trial | Trial | Trial | “Pass“ | Com- |
| Yes/No | Sample: | 1AP | 2AP | 3AP | Standard | ments |
| Yes | Pseudomonas | 0/60 | 0/60 | 0/60 | ≤1/60 | Pass |
| | aeruginosa | | | | | |
| | ATCC 15442 | | | | | |
| Yes | Salmonella | 0/60 | 0/60 | 0/60 | ≤1/60 | Pass |
| | enterica ATCC | | | | | |
| | 10708 | | | | | |
| No | Staphyllococcus | 0/60 | 0/60 | 0/60 | ≤1/60 | Pass |
| | aureus ATCC | | | | | |
| | 6538 | | | | | |
| Organic | | | | | | |
| Soil | | Log₁₀ | | | “Pass” | Com- |
| Load | Microbe(s) | Reduction in Microbe(s) | | | Standard | ments |
| No | Influenza A | ≥6.75 | ≥6.75 | N/A | minimum | Pass |
| | (H1N1) virus, | | | | of a 3 log | |
| | A/PR/8/34 | | | | reduction | |
| | strain, ATCC | | | | | |
| | VR-1469 | | | | | |
| No | Human | ≥5.00 | ≥5.00 | N/A | minimum | Pass |
| | coronavirus, | reduction | reduction | | of a 3 log | |
| | 229E strain, | | | | reduction | |
| | ATCC VR-740 | | | | | |

Example 6 demonstrates the successful use of the cleanser formulation disclosed herein. The cleanser formulation was dissolved in 360 ppm hardness water. Glass slides inoculated with the test microorganism were sprayed with the test formulation followed by being rinsed off. The slides were then cultured and observed for microbial growth. Microbial growth above EPA standards were counted as positive carriers. Slides with organic soil loads were treated with 5% fetal bovine serum.

### Example 7

Cleanser Embodiment-Bathroom Disinfectant

| Organic | | Number Confirmed | | | | |
|---|---|---|---|---|---|---|
| Soil | | Positive Carriers/ | | | | |
| Load? | Microbe(s) | Total Number Tested | | | “Pass” | Com- |
| Yes/No | Sample: | Trial 1B | Trial 2B | Trial 1A | Standard | ments |
| Yes | Pseudomonas | 0/60 | 0/60 | 0/60 | ≤1/60 | Pass |
| | aeruginosa | | | | | |
| | ATCC 15442 | | | | | |
| Yes | Staphyllococcus | 0/60 | 0/60 | 0/60 | ≤1/60 | Pass |
| | aureus ATCC | | | | | |
| | 6538 | | | | | |
| Yes | Salmonella | 0/60 | 0/60 | 0/60 | ≤1/60 | Pass |
| | enterica ATCC | | | | | |
| | 10708 | | | | | |
| Yes | Escherichia coli | 0/10 | 0/10 | N/A | ≤1/10 | Pass |
| | ATCC 11229 | | | | | |
| Organic | | | | | | |
| Soil | | Log₁₀ | | | “Pass” | Com- |
| Load | Microbe(s) | Reduction in Microbe(s) | | | Standard | ments |
| Yes | Influenza A | ≥4.50 | a ≥4.50 | N/A | minimum | Pass |
| | (H1N1) virus, | re- | re- | | of a 3 log | |
| | A/PR/8/34 | duction | duction | | reduction | |
| | strain, ATCC | | | | | |
| | VR-1469 | | | | | |

Example 7 demonstrates the successful use of the cleanser formulation disclosed herein. The cleanser formulation was dissolved in 360 ppm hardness water. Glass slides inoculated with the test microorganism were sprayed with the test formulation followed by being wiped off. The slides were then cultured and observed for microbial growth. Microbial growth above EPA standards were counted as positive carriers. Slides with organic soil loads were treated with 5% fetal bovine serum.

### Example 8

Cleanser Embodiment - Bathroom Disinfectant

| Analysis | Analysis Standard | Analysis Result | Method |
|---|---|---|---|
| Active Ingredient - | 0.05% | 0.05% | USP Thymol RS |
| Thymol | | | |
| Active Ingredient - | 0.10% | 0.10% | FTIR |
| Cupric Sulfate | | | |
| pH | 4.40-4.65 | 4.64 | pH meter |
| Dissolution Time | 6-8 minutes | 7:56 minutes | Internal |

A stable anhydrous disinfectant cleanser concentrate formulation of the present disclosure was prepared, and the pH and dissolution time were tested. A stable anhydrous disinfectant cleanser concentrate formulation has a dissolution time of under 8 minutes, namely 7 minutes 56 seconds, and a pH from about 4 to about 5, namely 4.64.

1. A stable anhydrous disinfectant cleanser concentrate formulation in a solid form, comprising a disinfectant, an acidic cleaner, a basic cleaner, and a surfactant.

2. The stable anhydrous disinfectant cleanser concentrate formulation of claim 1, wherein the disinfectant is thymol.

3. The stable anhydrous disinfectant cleanser concentrate formulation of claim 2, wherein the disinfectant is present in an amount from about 4% to about 25% by weight, based on the weight of the formulation.

4. The stable anhydrous disinfectant cleanser concentrate formulation of claim 2, wherein the disinfectant is present in an amount from about 4% to about 6% by weight, based on the weight of the formulation.

5. The stable anhydrous disinfectant cleanser concentrate formulation of claim 2, wherein the disinfectant is present in an amount from about 20% to about 25% by weight, based on the weight of the formulation.

6. The stable anhydrous disinfectant cleanser concentrate formulation of claim 2, further comprising copper sulfate.

7. The stable anhydrous disinfectant cleanser concentrate formulation of claim 6, wherein the copper sulfate is present in an amount from about 8% to about 12% by weight, based on the weight of the formulation.

8. The stable anhydrous disinfectant cleanser concentrate formulation of claim 6, wherein the copper sulfate is present in an amount about twice the amount of thymol by weight.

9. The stable anhydrous disinfectant cleanser concentrate formulation of claim 5, wherein the formulation does not include copper sulfate.

10. The stable anhydrous disinfectant cleanser concentrate formulation of claim 2, further comprising a preservative.

11. The stable anhydrous disinfectant cleanser concentrate formulation of claim 10, wherein the preservative is selected from sodium benzoate and potassium sorbate.

12. The stable anhydrous disinfectant cleanser concentrate formulation of claim 2, further comprising sorbitan caprylate.

13. The stable anhydrous disinfectant cleanser concentrate formulation of claim 12, wherein the sorbitan caprylate is present in an amount from about 0.1% to about 1% by weight, based on the weight of the formulation.

14. The stable anhydrous disinfectant cleanser concentrate formulation of claim 1, wherein the acidic cleaner is selected from citric acid, malic acid, fumaric acid, acetic acid, sodium acetate, and glycolic acid.

15. The stable anhydrous disinfectant cleanser concentrate formulation of claim 1, wherein the acidic cleaner is a coated citric acid that is designed not to react with the basic cleaner in the formulation.

16. The stable anhydrous disinfectant cleanser concentrate formulation of claim 1, wherein the basic cleaner is selected from sodium carbonate, sodium bicarbonate and any other alkali carbonates.

17. The stable anhydrous disinfectant cleanser concentrate formulation of claim 1, wherein the surfactant comprises an anionic and/or nonionic surfactant.

18. The stable anhydrous disinfectant cleanser concentrate formulation of claim 1, further comprising a binding agent.

19. The stable anhydrous disinfectant cleanser concentrate formulation of claim 1, which is substantially fatty acid free and/or substantially animal fat free.

20. The stable anhydrous disinfectant cleanser concentrate formulation of claim 1, wherein at least one of the acidic cleaner and the basic cleaner is present in an amount greater than that of the surfactant.

21. The stable anhydrous disinfectant cleanser concentrate formulation of claim 1, wherein the acidic cleaner and the basic cleaner together are present in an amount greater than that of the surfactant.

22. The stable anhydrous disinfectant cleanser concentrate formulation of claim 1, which is in the form of a tablet.

23. The stable anhydrous disinfectant cleanser concentrate formulation of claim 22, which is in the form of a tablet wherein the tablet is not tacky.

24. The stable anhydrous disinfectant cleanser concentrate formulation of claim 1, which is in the form of powder.

25. The stable anhydrous disinfectant cleanser concentrate formulation of claim 1, which is inserted in a wipe.

26. A method of preparing a tablet, comprising blending homogeneously the ingredients of claim 1 to form a mixture and compressing the mixture to form the tablet.

27. A method of using the tablet of claim 22 comprising (1) filling a spray bottle or vessel with water, (2) adding the tablet to the water-filled spray bottle or vessel, and (3) dissolving the tablet in appropriate amount of water.

28. The method of claim 27, further comprising applying the resulting solution to a surface to be cleaned.

29. A method of using the powder of claim 24 comprising diluting the powder in water at a powder to water ratio of greater than or equal to 1:1 (w/w) to form a paste, placing the paste on a surface to be cleaned either directly or through a rag or sponge, and rinsing the surface with water.

30. The method of claim 27, wherein the surface to be cleaned is a hand or a surface in a bathroom or kitchen.

31. A stable anhydrous disinfectant concentrate formulation in a solid form, comprising a disinfectant, an acidic cleaner, a basic cleaner, and a surfactant.

32. The stable anhydrous disinfectant concentrate formulation of claim 31, wherein the disinfectant is thymol.
